(19) United States
(12) Patent Application Publication
(10) Pub. No.: US 2011/0295056 A1
(43) Pub. Date: Dec. 1, 2011

(54) SYSTEMS AND METHODS FOR GASTRIC VOLUME REGULATION

(76) Inventors: Jeffrey L. Aldridge, Lebanon, OH (US); Michael S. Cropper, Edgewood, KY (US); Denzel Z. Herrera-Davis, Cincinnati, OH (US); Daniel F. Dlugos, JR., Middletown, OH (US); Jason L. Harris, Mason, OH (US); John V. Hunt, Cincinnati, OH (US); Prasanna Malaviya, Mason, OH (US); Jeffrey D. Messerly, Cincinnati, OH (US); Mark S. Ortiz, Milford, OH (US); Mark D. Overmyer, Cincinnati, OH (US); Galen C. Robertson, Durham, NC (US); Frederick E. Shelton, IV, Hillsboro, OH (US); Foster B. Stulen, Mason, OH (US); Suzanne Thompson, West Chester, OH (US); James W. Voegele, Cincinnati, OH (US); Lauren S. Weaner, Cincinnati, OH (US); Christopher W. Widenhouse, Clarksville, OH (US); Tamara S. Vetro Widenhouse, Clarksville, OH (US); David C. Yates, West Chester, OH (US); Mark S. Zeiner, Mason, OH (US)

(21) Appl. No.: 13/104,223

(22) Filed: May 10, 2011

Related U.S. Application Data
(60) Provisional application No. 61/348,271, filed on May 26, 2010.

(51) Int. Cl. A61F 2/04

(52) U.S. Cl. 600/37

(57) ABSTRACT

Devices and methods for modifying stomach volume include the formation of intragastric slots for wrapping one or more portions of the fundus therethrough with minimal interference with nerves and vasculature flow. Intragastric space occupying devices expand with environmental changes brought about by natural conditions inherent to the digestive cycle such as with changes in pH. Extragastric volume occupying balloons are placed into folded stomach sections. The balloons are fluidly coupled to external gastric filling devices. In yet another set of embodiments, methods and devices provide adjustable gastric volume reduction fundal wraps. In one embodiment, a device is placed in the fundus for Nissen fundoplication and permits postoperative adjustment to reach desired weight loss. Intragastric and extragastric balloons are optionally incorporated.

### PRIORITY

This application claims priority to U.S. Provisional Patent Application Ser. No. 61/348,271, entitled “Systems and Methods for Gastric Volume Regulation,” filed May 26, 2010, the disclosure of which is incorporated by reference herein.

### FIELD OF INVENTION

The present invention relates generally to systems and methods for regulating gastric volume.

### BACKGROUND OF THE INVENTION

Obesity is a major public health issue often leading to significant complications for an individual later in life. Obesity has been directly linked with the occurrence of additional health issues such as high blood pressure, stoke, arthritis, and diabetes among others.

Current options for surgical treatment of obesity include the use of gastric bands, gastrointestinal sleeves, and gastric bypass surgery. However, these methods for treatment of obesity often have undesirable side effects. For example, implanted gastric bands generally restrict passage of food, but do not reduce the actual volume of the stomach. Gastrointestinal sleeves are often difficult to secure within the gastrointestinal tract. These sleeves often are dislodged from their intended positions by the natural peristaltic motion of the stomach and further may be susceptible to breakdown by the highly acidic environment of the gastrointestinal tract. Further, gastric bypass surgery has been associated with numerous undesirable side effects such as diarrhea and nausea, as well as potentially fatal conditions such as liver failure. Also, Ghrelin is a hormone produced mainly by P/D1 cells lining the majority of the human stomach. These cells are distributed throughout the stomach and portions of the duodenum, but are highly concentrated in the area of the fundus and along the greater curvature of the stomach. Ghrelin, commonly called the hunger hormone, is associated with eating and fasting cycles in the body. It has been found that ghrelin levels increase before meals and decrease after meals. Further, it has been discovered that ghrelin levels in the plasma of obese individuals are typically lower than those in leaner individuals, while those suffering from the eating disorder anorexia nervosa typically have high plasma levels of ghrelin compared to both the constitutionally thin and normal-weight controls. These findings suggest that ghrelin plays a role in weight disorders. Additionally, increased Ghrelin levels have been linked to enhanced learning and memory, a reduction in stress-induced depression, and shorter sleep durations.

Therefore, there is a need for systems and methods to regulate food intake into the stomach that minimize unwanted side effects associated with known surgical obesity treatments and there remains a need for methods and devices for regulating the activation of ghrelin hormones within a stomach in order to treat weight disorders, to promote learning and memory functions, to treat stress-induced depression, and to promote healthy sleep duration.

Through recent research, it has been discovered that the enzyme Ghrelin-Octanoyl Acyl-Transferase (GOAT) mediates the control of ghrelin activation within the stomach. While dietary lipids serve as a substrate for GOAT which is used for acylation of circulating ghrelin, ghrelin acylation by GOAT may depend on the presence of specific dietary lipids. GOAT/ghrelin is a gastrointestinal lipid sensing system, yet the secretion and activation of ghrelin are two independently regulated processes. It is believed that the primary means for activating ghrelin is through the contact of the ghrelin producing cells of the stomach and/or intestines with stomach contents carrying the GOAT enzyme and dietary lipids necessary for activating ghrelin. The activated ghrelin, Human-Acyl-Ghrelin, moves from the stomach and/or intestines into the blood stream and its levels may be measured in the blood through known testing procedures. It has been found through testing that the Human Acyl-Ghrelin levels present in the blood stream decrease under fasting conditions. Accordingly, increased Human Acyl-Ghrelin levels in the blood stream may not reflect an empty stomach as previously thought; rather these increased Human Acyl-Ghrelin levels in the blood stream may actually be a signal indicating the availability of specific dietary lipids which may prepare the body for optimal nutrient partitioning and storage.

By blocking GOAT's access to ghrelin, ghrelin may be maintained in a non-activated state within the stomach, and may thereby reduce or eliminate hunger, promote learning and memory functions, treat stress-induced depression, and promote healthy sleep duration. Inversely, by facilitating GOAT's access to ghrelin, ghrelin may be maintained in an activated state within the stomach, and may thereby increase hunger or appetite, and alter healthy sleep duration. As may be appreciated, proper regulation of the activation of ghrelin hormones within a stomach may be utilized to treat or cure metabolic disorders, obesity, anorexia, depression, insomnia, learning or attention disorders, memory loss and the like.

### BRIEF DESCRIPTION OF THE DRAWINGS

The invention will be more fully understood from the following detailed description taken in conjunction with the accompanying drawings, in which:

FIG. 1A is a top view of a stomach having multiple incisions.

FIG. 1B is a top view of a fundus pulled through a slot in a stomach.

FIG. 1C is top view of a sutured fundal wrap.

FIG. 2A is a top view of a stomach having multiple incisions.

FIG. 2B is a top view of two fundal sections pulled through slots in a stomach.

FIG. 2C is a top view of a sutured, multiple slot fundal wrap

FIG. 3A is a top view of a stomach having multiple incisions.

FIG. 3B is top view of a sutured horizontal fundal wrap.

FIG. 4A is a top view of a stomach and internal volume occupying device.

FIG. 4B is a top view of a stomach and internal volume occupying device.

FIG. 4C is a top view of a fundus pulled through a slot over an internal volume occupying device.

FIG. 5A is a top view of a stomach and adjustable volume occupying device.

FIG. 5B is a top view of a fundus pulled through a slot over an adjustable volume occupying device.

FIG. 6 is a top view of a stomach and gastric balloon.

FIG. 7 is a perspective view of an auto-responsive space occupying device.

FIG. 8 is a chart of pH dependency on meal cycling.

FIG. 9A is a top view of a stomach and auto-responsive space occupying device.

FIG. 9B is a top view of a portion of an auto-responsive space occupying device.

FIG. 10 is a top view of a stomach and external volume occupying device.

FIG. 11A is a top view of a stomach and tubular expandable implant.

FIG. 11B is a top view of a stomach and tubular expandable implant.

FIG. 12A is a top view of a stomach and tailored expandable implant.

FIG. 12B is a side view of a stomach and tailored expandable implant.

FIG. 13A is a top view of a stomach and external balloon implant.

FIG. 13B is a top view of a stomach and external balloon implant.

FIG. 14A is a top view of a stomach and egg-shaped external balloon implant.

FIG. 14B is a top view of a stomach and bow-tie shaped external balloon implant.

FIG. 14C is a top view of a stomach with dual-egg external balloon implants.

FIG. 14D is a top view of a stomach and dog-bone external balloon implant.

FIG. 15A is a top view of a stomach and internal space occupying balloon.

FIG. 15B is a top view of a stomach and internal space occupying balloon.

FIG. 15C is a top view of a stomach and internal space occupying balloon.

FIG. 16 is a top view of a stomach and a gastric band and slot embodiment.

FIG. 17 is a top view of a fundoplication with a volume occupying device.

FIG. 18 is a top view of a fundoplication with a volume occupying device.

FIG. 19A is a top view of a fundoplication with a volume occupying device.

FIG. 19B is a top view of a fundoplication with a volume occupying device.

FIG. 20 is a top view of a fundal wrap.

### DETAILED DESCRIPTION OF THE INVENTION

The present invention discloses methods, system, and devices for gastric volume reduction (GVR). Exemplary embodiments herein are described to provide an overall understanding of the principles, structure, function, manufacture, and uses of the devices and methods included. Many specific examples of these embodiments are illustrated in the accompanying drawings. Those skilled in the art of gastrointestinal surgery and gastric device design will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be within the scope of the present invention.

Various exemplary methods and devices are provided for performing gastric volume reduction and nutrient flow regulation procedures. In a first set of embodiments, intragastric slots are used to wrap one or more portions of the fundus of a stomach therethrough. In a second set of embodiments, intragastric space occupying devices expand and contract to control the feeling of stomach satiation. These devices may change in volume due to externally induced stimuli or due to internal environmental changes brought about by natural conditions inherent to the digestive cycle. In a third set of embodiments, extragastric volume occupying balloons are placed into folded stomach sections and are similarly expanded or contracted by external stimuli such as being fluidly coupled to external gastric filling devices. In a fourth set of embodiments, adjustable gastric volume reduction fundal wraps are shown wherein a device is placed in the fundus to aid in the creation of a Nissen fundoplication and permit postoperative adjustment. Intragastric and extragastric balloons may optionally be incorporated.

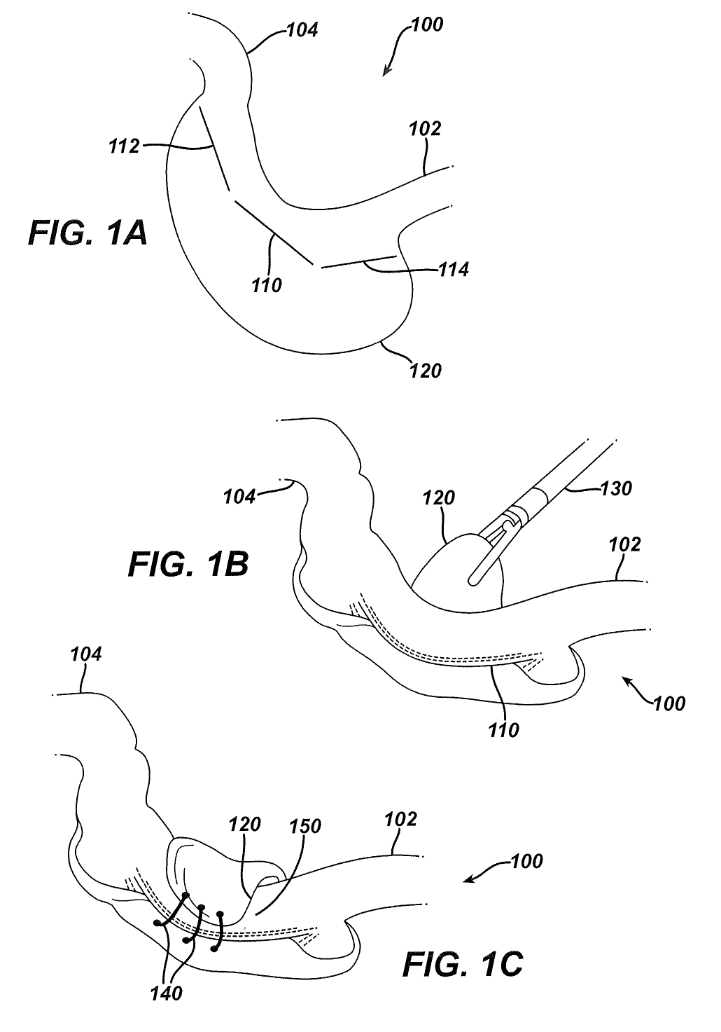

Turning now to FIG. 1A, a stomach 100 is shown attached between an esophagus 102 and a passage to the small intestine 104. In a first embodiment, a surgical procedure is used to reduce stomach volume and reroute nutrient flow away from the fundus and greater curvature by wrapping a portion of stomach 100 through a slot 110. Slot 110 is created by a single, substantially vertical incision made in stomach 100 to create a division in stomach 100 approximately parallel to the lesser curvature. Slot 110 may be created by any number of means envisioned by one of ordinary skill in the art. As a first example, and not by way of limitation, slot 110 is created by an incision in the body of stomach 100. In a second example, slot 110 is created by a Magenstrasse and Mill (M&M) procedure wherein a circular stapler creates an opening in the antrum or body of stomach 100. After creation of the opening, a linear cutter is then placed into the opening to create a divided staple line substantially parallel to the lesser curvature for the desired length. Non-limiting disclosures of the M&M procedure can be found in U.S. patent application Ser. No. 12/242,381, filed Sep. 30, 2008, entitled “Methods and Devices for Performing Gastroplasties Using Multiple Port Access”, and U.S. patent application Ser. No. 12/242,353, filed Sep. 30, 2008, entitled “Methods and Devices for Performing Gastrectomies and Gastroplasties” which are incorporated herein by reference in their entirety. Further, a non-limiting study on the operation is incorporated herein by reference in its entirety (Johnston et. al. The Magenstrasse and Mill Operation for Morbid Obesity; Obesity Surgery 13, 10-16). In a third example, a stapling device with selective stapling and cutting sections is used to form an initial opening. The stapling device creates a keyhole opening similar to the circular stapler without the excess costs and necessary port sizes associated with using circular staplers. Non-limiting disclosures of this procedure can be found in U.S. patent application Ser. No. 12/416,546, filed Apr. 1, 2009 entitled “Methods and Devices for Cutting Tissue” which is incorporated herein by reference.

As shown in FIG. 1B, after formation of slot 110 in the first embodiment, a fundus 120 of stomach 100 is wrapped about stomach 100 itself and pulled through slot 110 using a surgical instrument 130. Fundus 120 is optionally affixed to a portion of stomach 100 by sutures 140 as shown in FIG. 1C. Generally, in this first embodiment, fundus 120 is passed first through the newly created slot, behind and around the esophagus, and then attached back to either a narrowed section on the inferior curvature side of stomach 100, to another portion of fundus 120, or combinations of both. The final positioning and attachment are dependent upon the amount of tissue available and the operating surgeon's discretion as to appropriate placement. The wrapping maneuver described herein is similar in operation to a Nissen fundoplication. Further, it will be obvious to one of ordinary skill in the art that any number of alternative fixation means can be employed to affix fundus 120 to stomach 100 without the use of traditional sutures. As examples, the attachment may be completed with other fasteners such as staples, meshes, T-Tags, nitinol clips, barbed suture, and the like. Abrasion techniques may also be used to promote serousal-to-serousal healing as described in U.S. patent application Ser. No. 12/359,351, filed Jan. 26, 2009 entitled SURGICAL STAPLER FOR APPLYING A LARGE STAPLE THROUGH A SMALL DELIVERY PORT AND A METHOD OF USING THE STAPLER TO SECURE A TISSUE FOLD and U.S. Patent Application No. US 2009/0024144, filed Jul. 18, 2007 entitled HYBRID ENDOSCOPIC/LAPAROSCOPIC DEVICE FOR FORMING SEROSA TO SEROSA PLICATIONS IN A GASTRIC CAVITY which are incorporated herein by reference in their entireties.

Using this procedure, the stomach remains connected to the esophageal junction and near the pyrolus. A reduced gastric volume passageway or sleeve 150 is formed through which food can pass. An area of adjacent mucosa to mucosa contact is maintained in the wrapped portion of the stomach 100 to ensure ingested substances pass primarily through passageway 150 and deter contact of said ingested substances with the mucosa of the fundus and greater curvature. Sleeve 150 is the narrowed section on the lesser curvature side of stomach 100 as shown in FIG. 1C and functions as a self-adjusting band around stomach 100. As food passes through the esophagus toward the volume reduced stomach, sleeve 150 fills. As the wrapped section expands with eating, pressure is applied to the modified portion of stomach 100 thus creating a gastric banding-like effect. In addition, as the reduced volume fills with food and breakdown of the food continues in digestion, natural muscular contraction initiates and applies additional pressure to the wrapped portion of stomach 100. The additional pressure from digestion creates a feeling of being “full” faster thereby causing the patient to consume less due to a greater feeling of satiation. Since the wrapped stomach also contains food that has been restricted by a tightened exit, the food will take longer to exit and the effect is that the patient will fill “full” longer and experience a longer period of satiation.

This procedure offers several benefits. First, it can be tailored to leave nerve endings and vasculature in place as desired. Another benefit is that the stomach provides self-regulating and self-adjusting aspects to regulate consumption and satiation. If the patient attempts to eat excessively, the modified stomach 100 provides the ability to squeeze off the potential stomach volume which in turn reduces the patient's ability to continuously eat. Eventually, the system self-restricts and excess will back up into the esophagus near the esophageal junction. This backup will create an additional pressure on the chest of the patient causing a feeling much like that of experiencing heartburn.

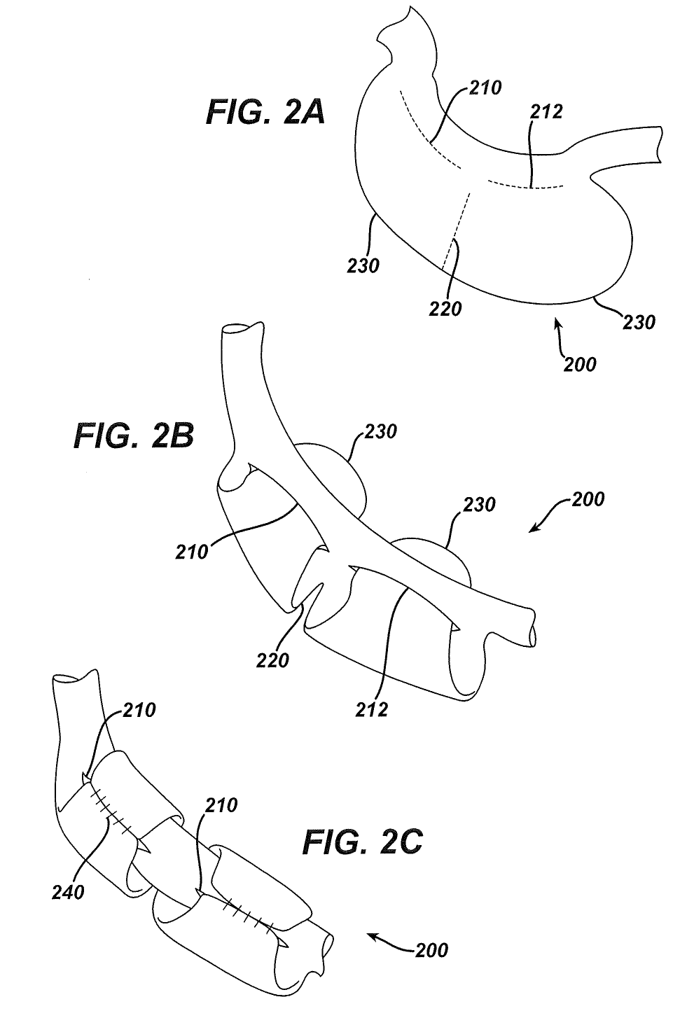

Turning now to FIG. 2A, a second embodiment is shown in a stomach 200 wherein a multiple slots 210, 212 are created. A horizontal slot 220 is further created through a stomach fundus 230 such that wrapping is more easily managed using discrete portions of the fundus 230. As shown in FIG. 2B, separate portions of fundus 230 are pulled individually though the slots 210, 212. The fundus portions 230 are then wrapped about stomach 200 as shown in FIG. 2C and then affixed by sutures 240 to themselves or alternatively to other portions of stomach 200. Means for affixing the fundus portions 230 include sutures, staples, and aforementioned means as described with respect to the prior described embodiments.

The second embodiment offers the patient and surgeon several noteworthy advantages. Nerve endings and vasculature are less restricted using a multiple slot method. Further, the division of fundus 230 into two sections simplifies formation of the fundal wrap by dividing the weight of fundus 230 into more manageable fundal portions, as two leaves of the stomach would be inserted into slots 210 and 212 independently thereby lessening the amount of stomach 200 that would have to be managed by the surgeon at any one time. In addition, this approach allows differing amounts of wrap tightness to be applied to optimize stomach size. Yet another benefit is that this multiple slot embodiment minimizes the size of creases in stomach 200 since smaller portions of stomach 200 are folded.

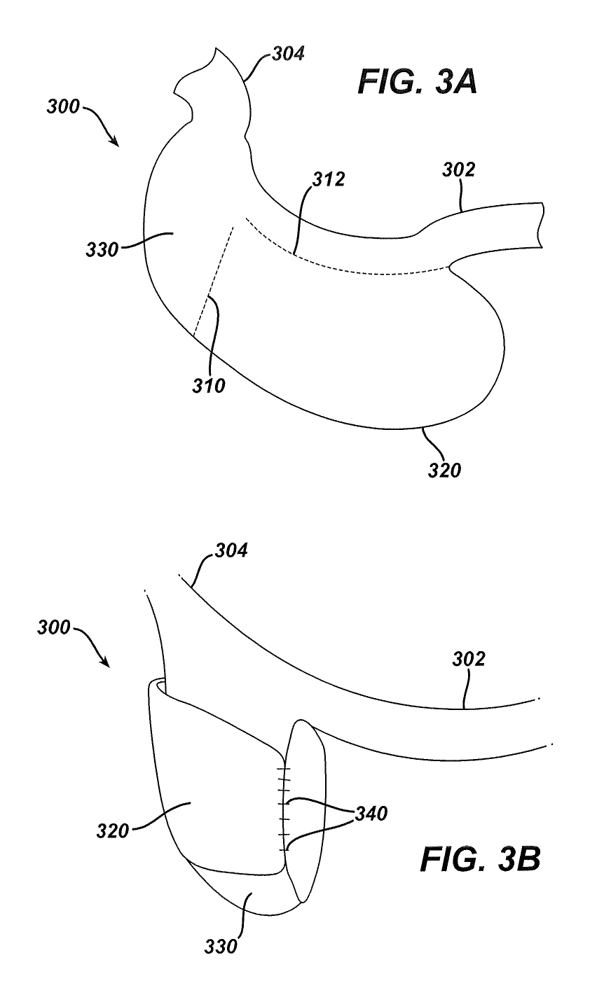

In FIG. 3A, a third embodiment is shown in a stomach 300 having an esophagus 302, a fundus 320 and an antrum 330. In this embodiment a horizontal wrap is formed by creating a horizontal slot 310 below a portion of stomach 300 near an entrance 304 to the small intestine as shown in FIG. 3A. Horizontal slot 310 is created approximately one-half to two-thirds down the length of fundus 320. In addition, a vertical transsection 312 is created from the angle of His down along the lesser curve of stomach 300 sufficient to free up fundus 320. The freed fundus 320 is then folded down through horizontal slot 310 and then around antrum 330 before securing by attachment means 340.

As illustrated in FIG. 3B and shown by example, fundus 320 is folded back up to slot 310 on an anterior side and is attached by means of staples, meshes, T-Tags, and the like. It is noteworthy that this embodiment permits cutting of the vagus nerve which has been shown in numerous studies to have positive effects on weight loss.

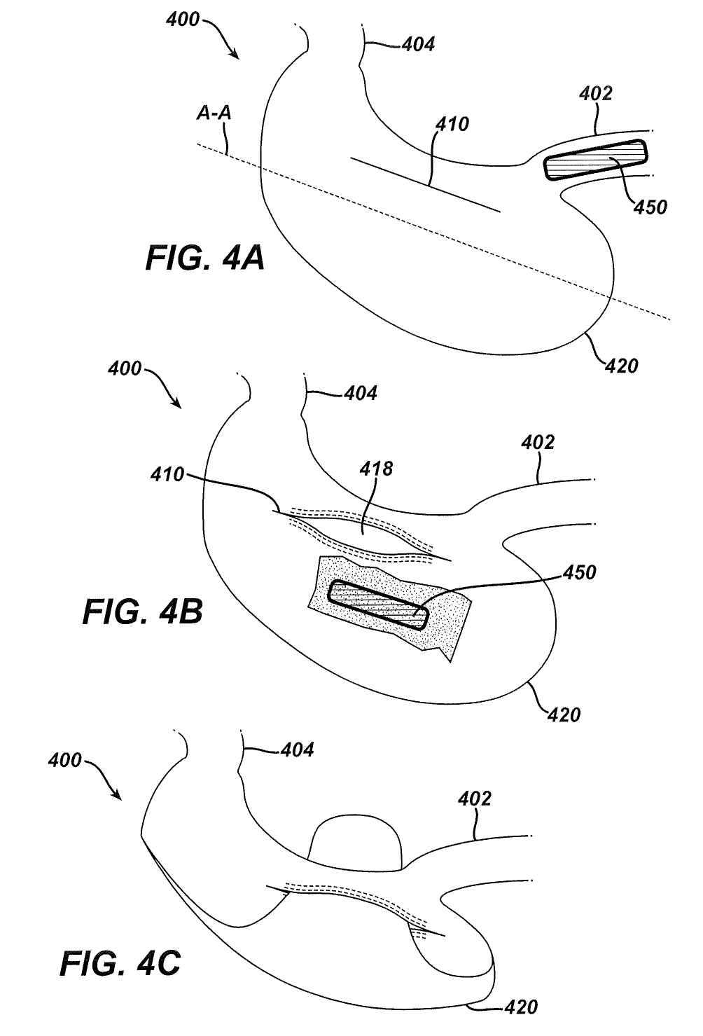

Turning now to FIGS. 4A-4C, in yet another embodiment, a variation of the aforementioned embodiments includes a volume occupying device in a pocket area of the fundus area of the stomach created by the wrap that is internal to the stomach wall. The volume occupying device, or bulking agent, is used to simultaneously stretch the stomach and to provide the sensation of satiation. The volume occupying device is also used to limit the residence volume of food in the stomach and also to limit food intake.

As shown in FIG. 4A, a volume occupying device 450 may be introduced to a stomach 400 transorally via passage through a connecting esophagus 402. Once introduced, volume occupying device 450 is positioned centrally to stomach 400 near a linear axis A-A shown in FIG. 4A across which stomach 400 is later folded. After positioning, a vertical slot 410 is created in stomach 400 before fundus 420 of stomach 400 is folded through slot 410 such that volume occupying device 450 is maintained in stomach 400. Fundus 420 may be subsequently wrapped across the stomach 400 and secured by attachment means as previously described.

By combining an adjustable volume occupying device with the aforementioned slot and wrap procedures, the amount of restriction provided by these procedures can be adjusted post-operatively. The capability of post-operative adjustment allows the procedure to vary in restriction accuracy through fine-tuning of the gastric passage volume without the need for additional surgical procedures. For example, delaying the inflation of an adjustable volume occupying device allows post-operative swelling to decrease. Further, the use of an adjustable volume occupying device eliminates the potential for problems that are typically caused by food blockage from dislodged gastric implants.

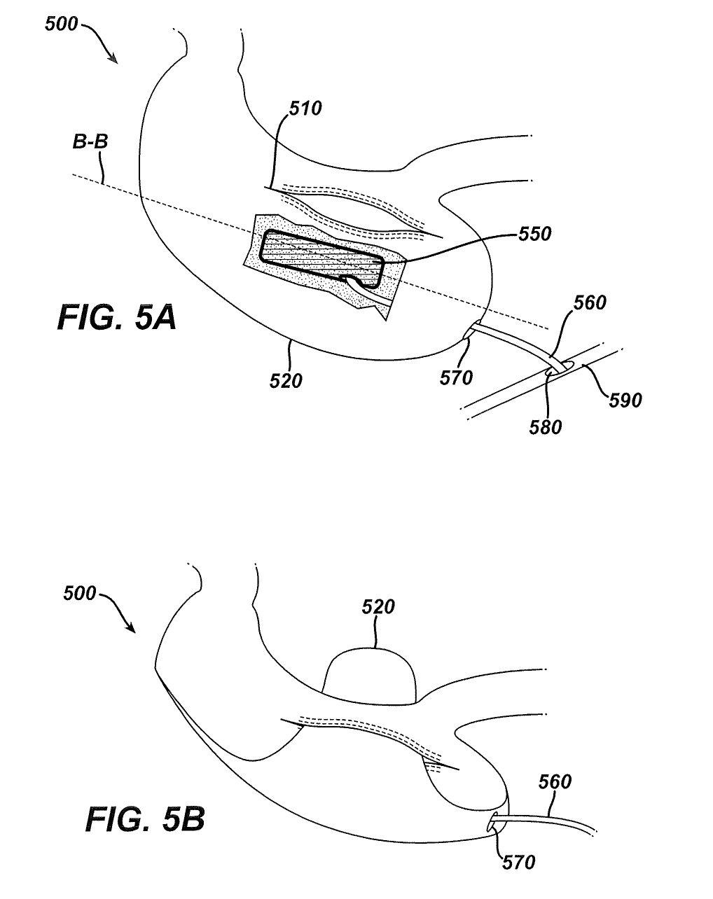

In an exemplary embodiment, the adjustable volume occupying device is a gastric balloon 550 as shown in FIGS. 5A and 5B. Balloon 550 is shown within a fundus 520 of a stomach 500 and balloon 550 is approximately centered across an axial fold line B-B. After placement of balloon 550 in stomach 500, fundus 520 is folded through a vertical slot 510 formed by methods described in prior embodiments. After fundus 520 is folded through slot 510, it may be wrapped about stomach 500 and secured by attachment means to other portions of fundus 520 or to stomach 500.

As seen in FIG. 5A, gastric balloon 550 may include a fill tube 560 that extends from balloon 550 through an opening 570 in stomach 500 and further through an adjustment port 580 in an exterior surface 590. Using fill tube 560, the volume of the balloon can be expanded or contracted via adjustment port 580 or by means of a pump or other fluidic control between balloon 550 and an external reservoir (not shown).

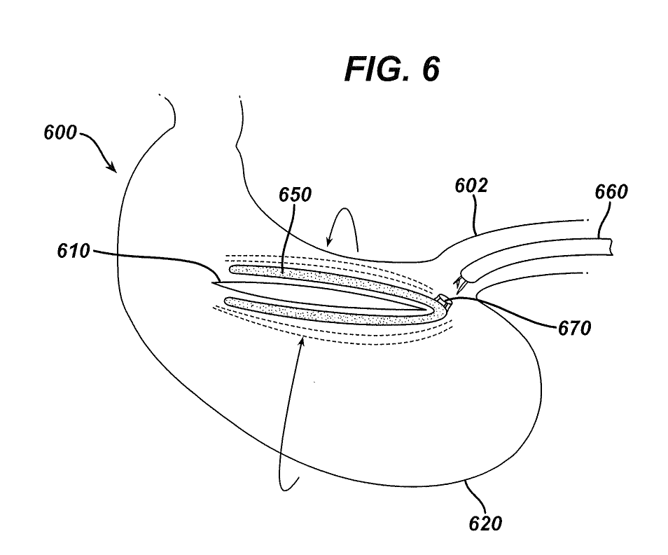

Turning now to FIG. 6, another gastric balloon embodiment is provided. Here, potential difficulties associated with precise alignment of a gastric balloon are solved by the use of a saddle-wedged balloon 650 position about a slot 610 formed in a stomach 600 having a fundus 620. Saddle-wedged balloon 650 is positioned on both sides of slot 610 as seen in FIG. 6 such that slot 610 provides a stabilizing element to affix the position of balloon 650. In this embodiment, fundus 620 may be similarly pulled through slot 610 and affixed to stomach 600 or fundus 620 as previously described with respect to other embodiments of the disclosure. In addition, a fill tube 660 may be provided to regulate the volume of balloon 650 by attachment to a fill tube port 670 as shown in FIG. 6. Fill tube 660 may be passed intra-orally down an esophageal passageway 602 or alternatively brought in through an exterior surface as described with respect to previous embodiments.

In still another embodiment of an internal volume occupying device is an auto-responsive space occupying device. An auto-responsive space occupying device changes volume as a function of consumption so that the device expands to induce volume reduction and may also apply pressure within the stomach to induce a feeling of satiation when the patient is hungry. The device may apply pressure when eating would adversely affect stomach tissue relaxation. This auto-responsive effect prevents stretching and relaxation of stomach tissues associated with a constant volume device that would undesirably decrease the level of its effectiveness the longer the constant volume device remained in place. Further, complications arising from blockages potentially caused by a constant volume device are alleviated.

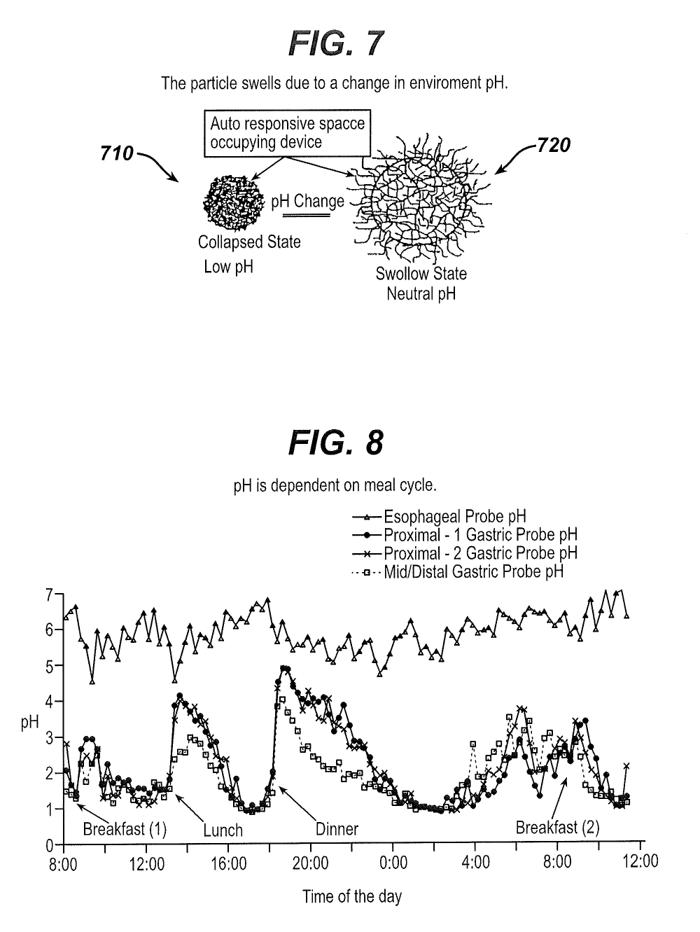

In one example of an auto-responsive space occupying device shown in FIG. 7, the device is a pH sensitive hydrogel balloon. Several polymers are suitable for this purpose and are otherwise known as environmentally sensitive hydrogels. These polymers change physical properties as a function of environmental conditions within the stomach. Triggering environmental conditions may include chemical changes, temperature changes, pH changes, and pressure changes. The polymers may increase or decrease in volume as changes in these environmental conditions occur. A space occupying device may be constructed from individually sensitive polymers or one or more combinations of these polymers. In the example of FIG. 7, an environmentally sensitive polymer is shown in a collapsed state 710. The polymer 710 is responsive to pH changes in the stomach and swells to expand, as shown by reference indicia 720, in direct response to increases in pH and in particular to changes in pH caused when consumption of food is initiated. The expansion of the device 710 driven by the change in pH increases the volume of the device 710 inside the stomach and consequently causes a feeling of satiation within the patient as food intake begins. After a period of time when food intake has ceased, the physiology of the patient assumes a lower pH and the device relaxes by returning to a smaller volume.

In yet another embodiment of the invention, one or more environmentally sensitive hydrogels are placed into a single container or pouch (not shown). The container is constructed of a material that is resistant to the grinding and churning mechanisms of digestion, gastric motility, and also enzymatic attack by digestive enzymes. Suitable materials for this container would include some metals such as nitinol, polymeric materials such as polyesters including polyethylene terephthalate, polyolefins including polyethylene, polypropylene, and copolymers thereof, silicone elastomers such as polymers based on dimethyl siloxane and also including materials functionalized with phenyl or fluoro groups to include phenyl silicones and fluorosilicones, fluoropolymers including expanded PTFE, PTFE copolymers, and also terpolymers synthesized with monomer groups containing tetrafluoroethylene (TFE), hexafluoropropylene (HFP), vinylidine fluoride (VDF), and others that make up the class of materials known as fluoropolymers and fluoroelastomers, and also other materials that would meet the environmental resistance characteristic requirements described herein.

In an example of the container or pouch, the container is porous to gastric fluids to permit these fluids to freely enter and leave the container freely such that when a change in the physiological conditions occurs related to consumption of food, these changes also occur within the container. In another example, the container is constructed as a series of multiple environmentally sensitive hydropolymers that interact together to cause changes in the occupied volume.

Where the changes in volume are related to pH in the aforementioned examples and embodiments, physiological changes of the digestive system between fasting and consumption are described in terms of changes in gastric acidity as measured by pH (the inverse log of hydronium ion concentration or log (1/[H+]). The pH scale spans from 1 (acidic) to 14 (basic) with 7.0 representing a neutral pH. During a fasting state, the stomach pH is typically acidic with a low pH value. When meal digestion occurs, there is a buffering of intragastric acidity with an elevation of gastric pH.

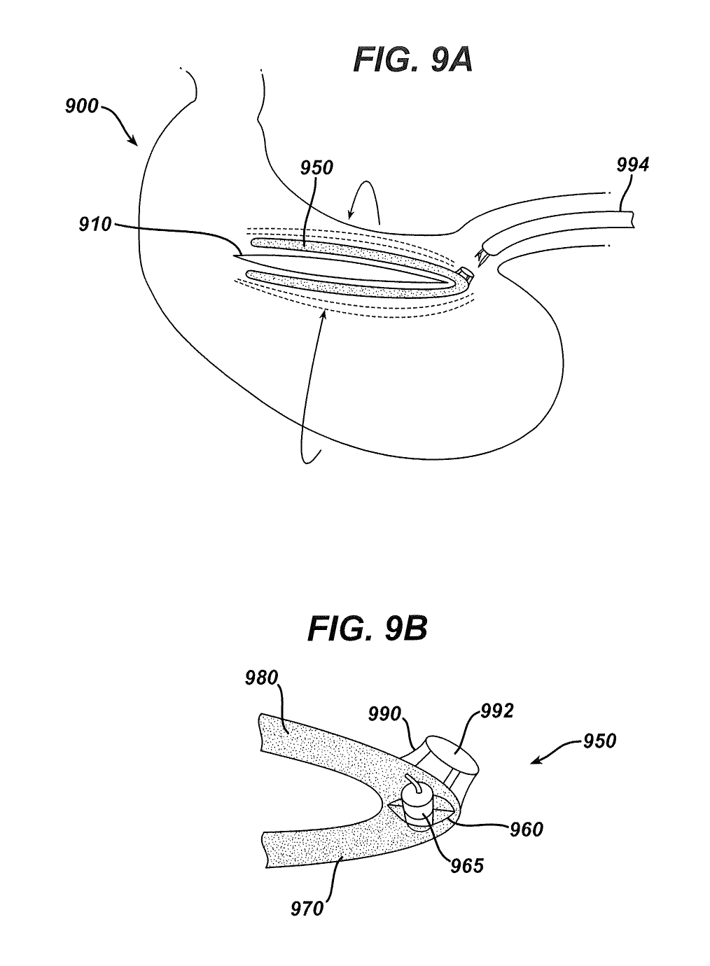

As seen in FIG. 8, monitored and measured changes in gastric pH are shown taken from Simonian et al. (Simonian, H. P., Vo., L., Doma, S. Fisher R. S., Parkman, H. P., Regional Postprandial Differences in PH Within the Stomach and Gastrointestinal Junction, Digestive Diseases and Sciences, 2276-2285, 50 (12), 2005). As seen in FIG. 8, the change in pH occurs rapidly with the initiation of consumption as food enters the stomach. This change also occurs even with the secretion of gastric acids continuously during consumption. The buffering capacity of foods is sufficient to provide a significant change in gastric pH.

In still another embodiment, an intragastric pH sensor is placed within the stomach and triggers a change in volume of an intragastric space occupying device. A wrapped stomach geometry, as described with earlier embodiments and shown in FIGS. 1C and 2C, allows a much smaller configuration for the low volume state which is beneficial for endoluminal placement. Extragastric devices may also be used as reservoirs from which volume is added intragastrically via transgastric pathways. The auto-responsive space occupying devices described above may be used as internal devices incorporated into the fold of the slot wrap procedures described with respect to FIGS. 1A-3B. The intragastric pH sensor serves as a regulating element for control of the system.

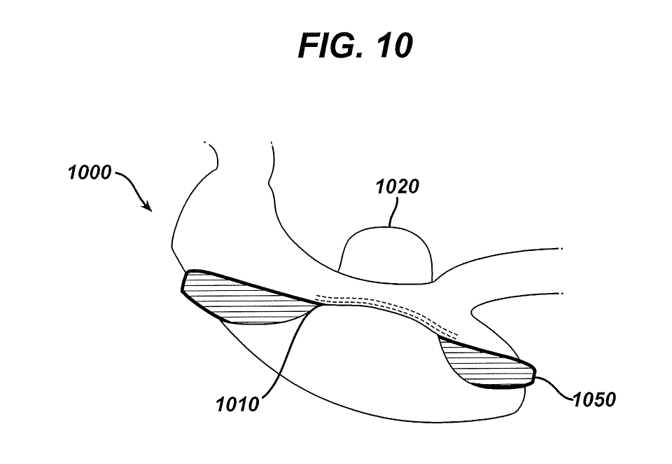

Turning now to FIGS. 9A and 9B, in still another embodiment, the auto-responsive space occupying device would involve an auto adjust band wedged between the upper portion of a slot 910 in a stomach 900 and the angle of His as previously described. In this embodiment however, the legs of a balloon 950 are divided into two chambers by a partition wall 960 which keeps fluid in a reservoir 970 separate from fluid in a main alimentary chamber 980. An implantable pump 965, such as a Debiotech nano-pump, could be fixed in the partition in fluid communication with both sides of balloon 950 and disposed to move fluids back and forth between the chambers, adjusting the size. By detecting pressure in the alimentary chamber 980, the system pumps fluid from the reservoir 970 into the alimentary chamber 980 as needed. Filling of reservoir 970 is accomplished by providing a fill port 990 having a septum 992 in balloon 950 for juncture with a fill tube 994.

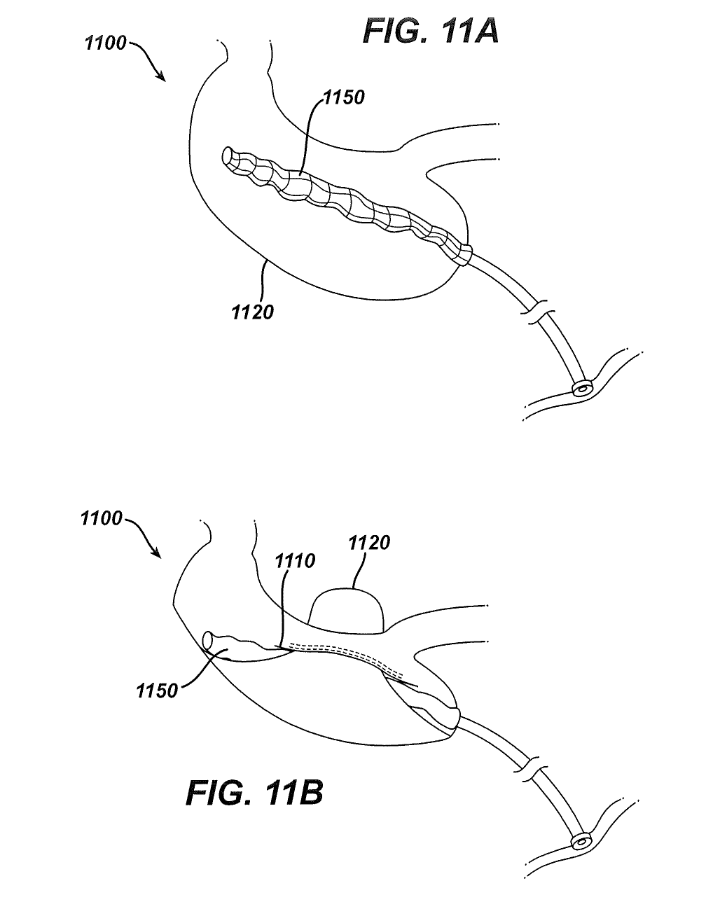

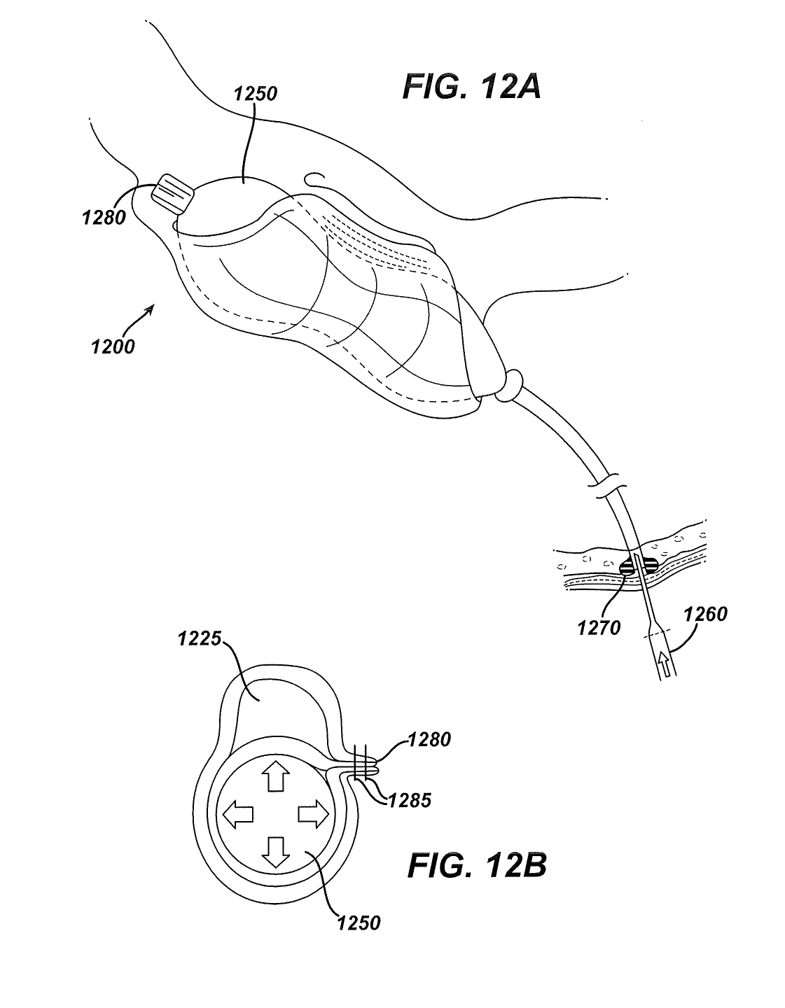

Turning now to FIGS. 10-12, in another variation of the slot wrap procedures described above, a volume occupying device is provided in a pocket area of a slot that is external to a stomach. In an example of such an application, the external occupying device is an external saline balloon implant 1150. The implant 1050, 1150 is used in various forms. For example, the implant 1050, 1150 may be inserted onto a stomach fundus 1020, 1120 as shown in FIGS. 10 and 11A prior to folding of the fundus through a slot 1010, 1110 formed in stomach 1000, 1100 at the time of the surgical procedure. As seen in FIG. 11B, the implant resides in a pocket formed by folding of fundus 1120 through slot 1110.

As seen in FIGS. 11A and 11B by way of example, the implant is a tubular expandable implant. The implant is deflated to its smallest condition at the time of implantation and after the tissue heals following the surgical procedure, the expandable implant is adjusted over time to facilitate long term adjustability of the procedure. No cutting is required to facilitate adjustability. Alternatively, the implant may be inserted laparoscopically using a trocar.

Generally, the implant may have a fill port 1160 to permit subcutaneous adjustments similar to that of an adjustable gastric band. Fill port 1160 is attached to expandable implant 1150. Implant 1150 may be stitched inside the fold formed at slot 1110 such that implant 1150 is effectively contained within the stomach boundaries. Although no cutting of stomach 1100 is required to place the implant 1150, the net result of the procedure is that implant 1150 is inside stomach 1100.

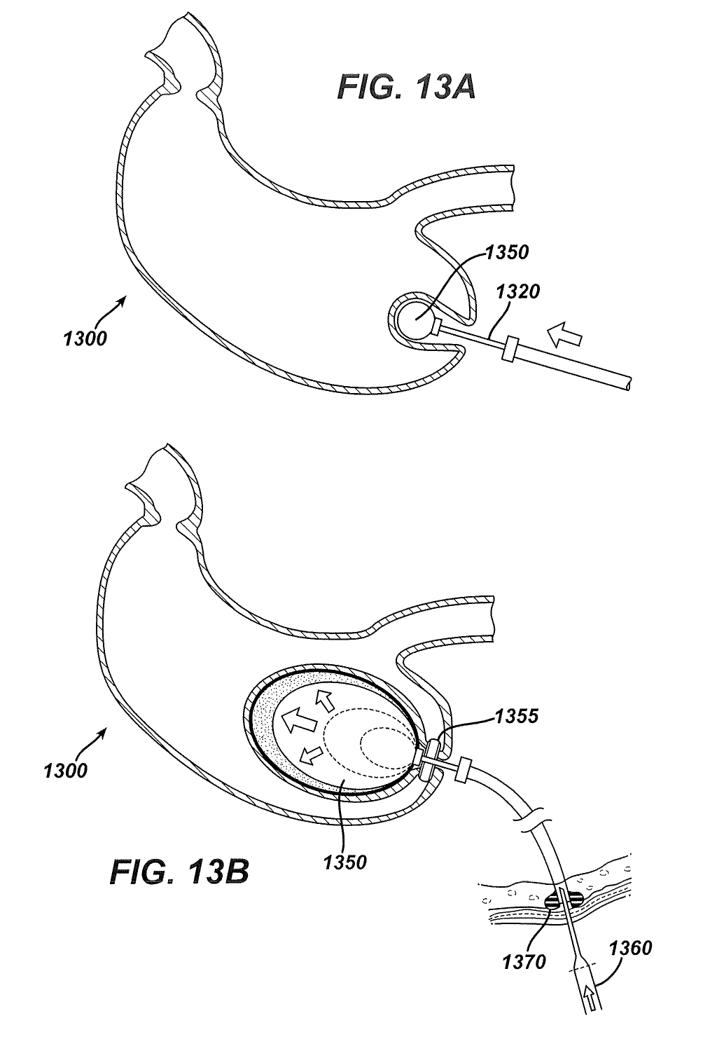

As seen in FIGS. 12A and 12B, an implant 1250 may have a tailored device shape. The expandable implant 1250 is custom-designed by a surgeon to apply varying amounts of pressure along the axial length of implant 1250 as implant 1250 is expanded and contracted. This permits selective application of pressure to points in the stomach to facilitate feelings of satiation within the patient with greater precision. In some embodiments, expandable balloon 1250 is inflated and deflated by means of a saline injector 1260 inserted through a transcutaneous valve 1270. Optionally expandable implant 1250 may include an anchoring element 1280 for facilitate fixation where implanted. As examples, anchoring element 1280 may include one or more protrusions or may incorporate staples 1285 at one end of implant 1250 for regulation of a restricted area 1225 of stomach 1200.

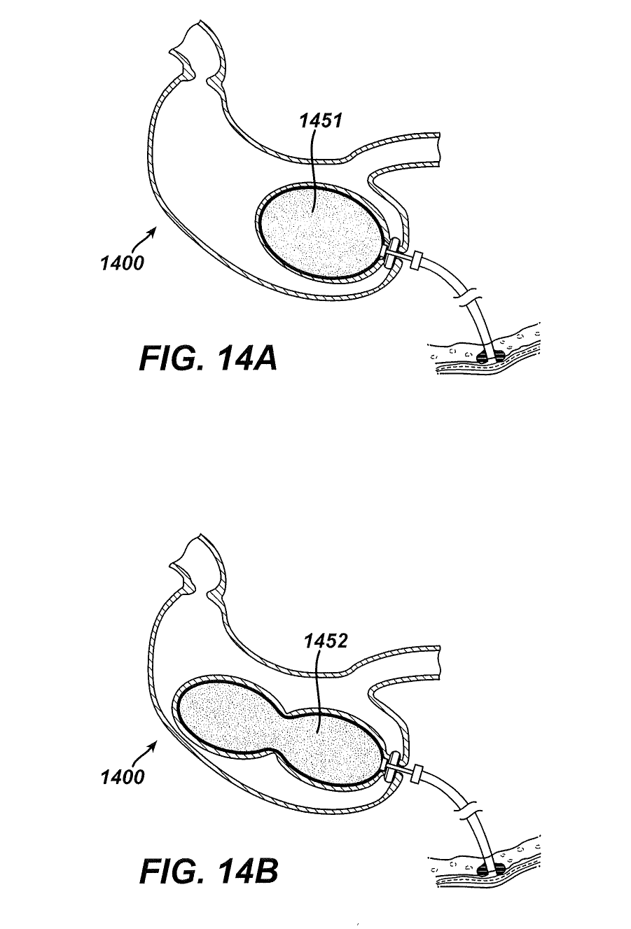

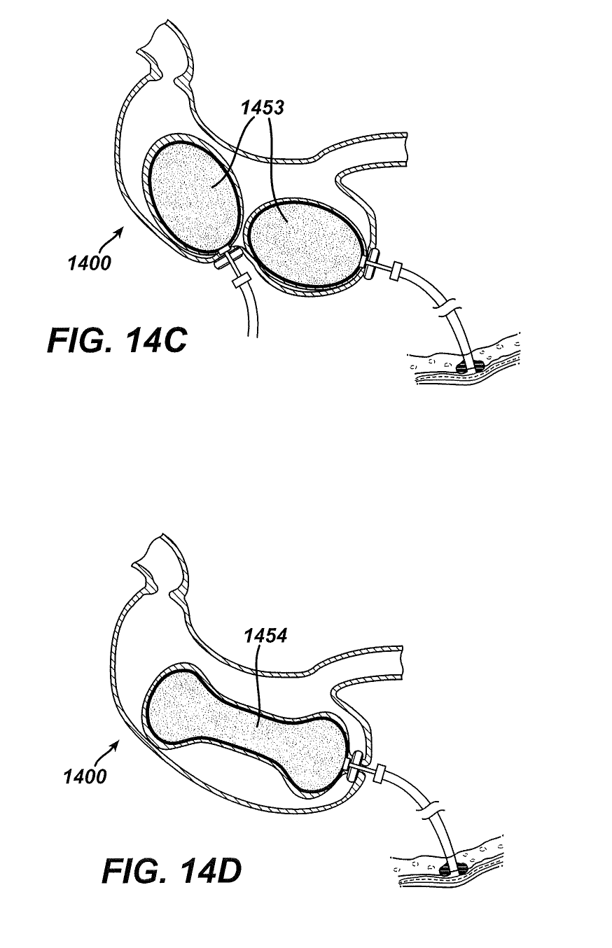

Turning now to FIGS. 13-14, additional embodiments of the gastric volume reduction systems and methods of the present invention are depicted wherein external volume occupying devices are attached to outer portions of stomach walls to reduce effective stomach volume.

In a first example as seen in FIGS. 13A and 13B, an external balloon 1350 is introduced into a stomach 1300 by a push rod 1320. Balloon 1350 is fixed into place by an internal clip 1355 before balloon 1350 is expanded by means of injection. The injector may be a saline injector 1360 inserted through a transcutaneous port 1370. Balloon 1350 is expanded to apply varying amounts of pressure to different points along stomach 1300 defined by the shape of expanding balloon 1350. Balloon 1350 may be custom designed by the surgeon to selectively apply pressure along different points of stomach 1300 without resection or cutting.

As shown in FIGS. 14A-D, any number of customized shapes may be preformed for the external balloon to facilitate tailored application of pressure along the perimeter of the stomach. In a first example shown in FIG. 14A, an egg-shaped external balloon 1451 is used to apply pressure to a perimeter of a stomach 1400. Egg-shaped external balloon 1451 applies pressure approximately evenly across the surface of stomach 1400. In an alternative design shown in FIG. 14B, a bow-tie-shaped external balloon 1452 applies pressure centered at two desired points along stomach 1400. In another alternative design, multiple egg shaped balloons 1453 are used to provide individualized control of pressure to points of stomach 1400 using separate control elements for each balloon as see in FIG. 14C. In yet another example, a dog-bone-shaped external balloon 1454 applies pressure centered at two desired points along stomach 1400. Although the examples presented in FIGS. 14A-D represent potential configurations of the external balloon design of the present invention, it will be well-recognized by a person of ordinary skill in the art of gastric device design that any number of alternative shapes and design patterns for applying pressure may be used to achieve pressure emphasis on various parts of the stomach in a controlled manner as described herein and the examples presented are in no way limiting of the geometrical configurations possible.

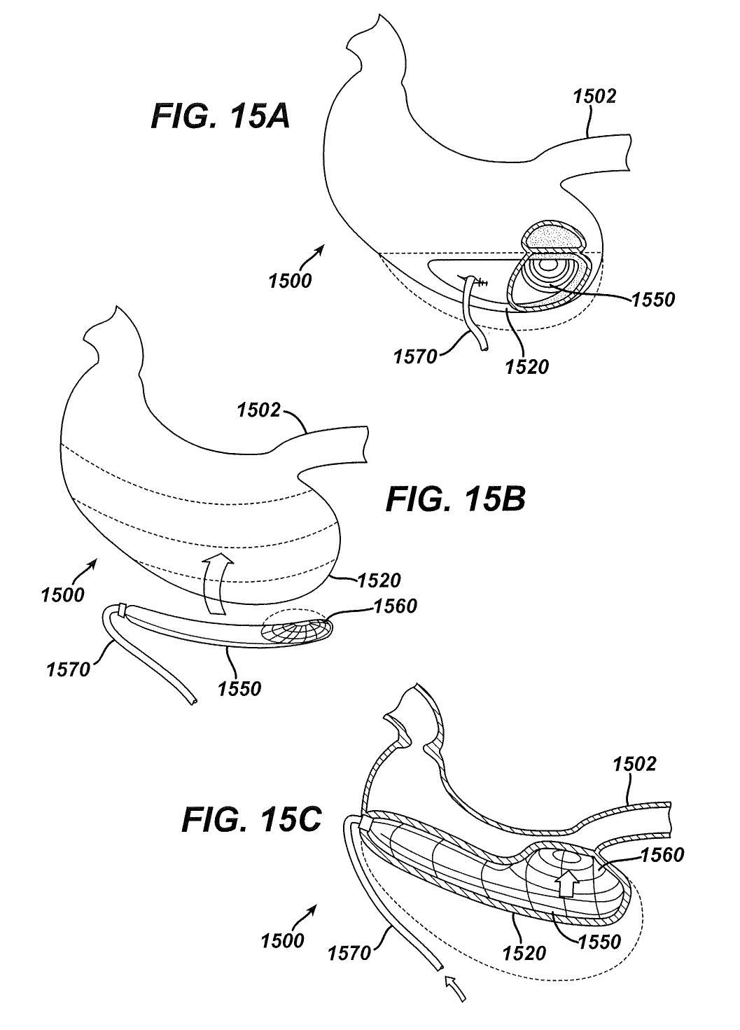

FIGS. 15A-C show an alternate embodiment of a space occupying balloon in which the balloons are designed for customized expansion in selected portions of the balloon. In FIG. 15A, an internal space occupying balloon 1550 in inserted into a plication of a stomach 1500 having a fundus 1520 and attached to an esophagus 1502. Balloon 1550 may comprise a transgastric valve 1570 to facilitate inflation and deflation of balloon 1550 by means of an external control device (not shown). In FIGS. 15B and 15C, balloon 1550 is shown comprising an expanding portion 1560 that expands to apply pressure disproportionately to a desired location of fundus 1520 or to contract and thereby release the selectively applied pressure. In FIG. 15C, balloon 1550 is shown implanted into fundus 1520 of stomach 1500 wherein balloon 1550 is positioned to selectively apply additional pressure near an area of esophagus 1502. Other geometrical configuration are readily envisioned by one of ordinary skill in the art to selectively apply pressure to any number of regions inside and throughout stomach 1500 and the example provided by FIGS. 15A-C are in no way intended to be limiting of the balloon pressure configurations possible.

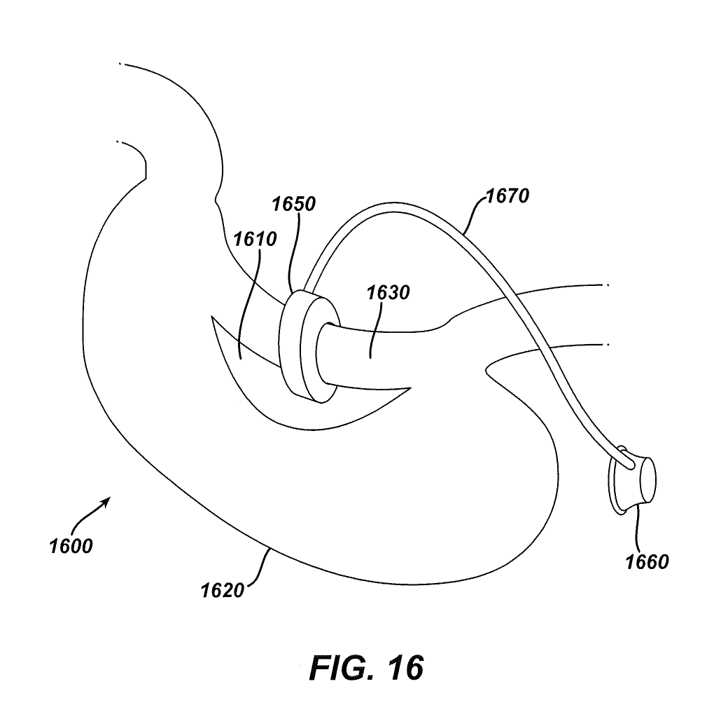

In another embodiment for regulating gastric volume, FIG. 16 shows the application of a gastric band 1650 through a slot 1610 formed in a stomach 1600 having a fundus 1620 such that gastric band 1650 encircles a sleeve portion 1630 of stomach 1600 opposite fundus 1620. Gastric band 1650 may be inflated or deflated by actuation of an external fill port 1660 comprising a transcutaneous fill tube 1670 that connects an external fluid reservoir (not shown) to gastric band 1650. In one embodiment, gastric band 1650 is placed around sleeve portion 1630 prior to wrapping stomach 1600 through slot 1610 and around gastric band 1650. This provides adjustability to the alimentary chamber of stomach 1600 where food flows. Gastric band 1650 may be adjusted using fill port 1660. In slot 1610, gastric band 1650 is prevented from slipping and rendering the need for a gastro-gastric wrap unnecessary. Further, gastric band 1650 may be moved cylindrically, lowering the pressure on the tissue and also reducing the likelihood of erosion of gastric band 1650. An added benefit is that placement of gastric band 1650 directly on sleeve portion 1630 creates a much more direct and predictable effect on the volume of stomach 1600 when actuated.

Additional applications of the gastric volume regulation devices and methods described in present disclosure include the application to partial or full fundoplication procedures. In a fundoplication, the fundus of the stomach is wrapped partially or fully around the esophagus and attached to the anterior side of the outer stomach wall. A fundoplication produces desired weight loss by reducing the effective stomach size which in turn reduces the required amount of food to provide a feeling of satiation or fullness.

A variation on the fundoplication procedure for weight loss is to include an internal volume occupying device as described with respect to prior embodiments in the fundal wrap. The volume reducing effect of the procedure is adjustable post-operatively by the inclusion of the volume occupying device. Alternatively, the fundus may be plicated as described in U.S. Application Publication No. US 2009/0024144, filed Jul. 18^{th}, 2007 entitled HYBRID ENDOSCOPIC/LAPAROSCOPIC DEVICE FOR FORMING SEROSA TO SEROSA PLICATIONS IN A GASTRIC CAVITY which is incorporated herein by reference in its entirety.

The plications described in U.S. Application Publication No. US 2009/0024144 and U.S. patent application Ser. No. 12/359,351 can be secured using many different fastening means including but not limited to the use of sutures, staples, nitinol clips, T-Tags, barbed suture, and adhesives (e.g., from the class of implantable cyanoacrylates, etc.). One non-limiting example of a nitinol clip is a larger version of the Coalescent Surgical U-Clip™ available from Medtronic, Inc., Minneapolis, Minn. In this example a flexible member of nitinol wire is held in a deformed configuration and passed through tissue. Once through the tissue, the wire is released and allowed to obtain an undeformed configuration wherein the shape is a closed loop. This closed loop can serve as a tissue securing feature. In another non-limiting example, a barbed suture may be used to secure the fold. Barbed sutures contain tissue securing features that allow passage of the suture through a tissue in one direction while resisting or preventing passage of the suture in another direction so as to minimize or eliminate the need for knot tying. A non-limiting example of a barbed suture is found in U.S. Application Publication No. US 2007/0005110, filed Jun. 29, 2005 entitled “Braided Barbed Suture” which is incorporated herein by reference in its entirety. Specialized appliers (devices, needles, etc.) for the aforementioned fasteners may be fabricated and/or used for performing a plication procedure (e.g., gastric plication about the greater curvature of the stomach following dissection of the greater omentum and division of the short gastric vessels, etc.), such as combining a barbed suture with an existing suturing device such as the Suture Assistant™ available from Ethicon Endo-Surgery, Inc., Cincinnati, Ohio. These plication techniques may be applied to the fundoplication procedures as well as other procedures described herein.

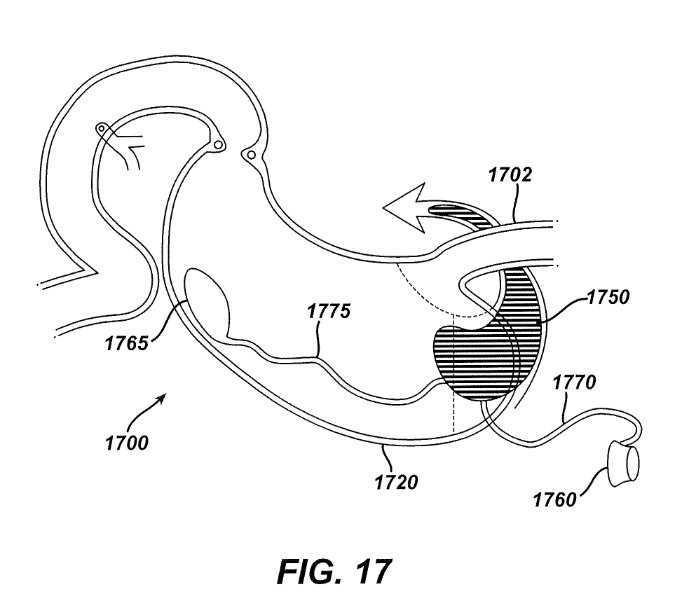

Turning now to FIG. 17 in a first embodiment of a fundoplication procedure, a stomach 1700 having a fundus 1720 is shown attached at one end to an esophagus 1702. Fundus 1720 is seen having an internal volume occupying device 1750. Volume occupying device 1750 may be inserted laparoscopically into stomach 1700 prior to a fundoplication procedure. During fundoplication, fundus 1720 is wrapped around esophagus 1702 as shown in FIG. 17. After wrapping, fundus 1720 is optionally secured to an opposite or anterior portion of stomach 1700. After fundoplication, volume occupying device 1750 can be expanded and contracted by internal or external methods. In a first method, an extra-luminally filled port 1760 located outside the body of the patient is used to expand or contract internal volume occupying device 1750 via a transgastric fill tube 1770. In a second method, an internal reservoir 1765 is position in the stomach 1700 to provide fluids to internal volume occupying device 1750 by way of an intragastric fill tube 1775. Both methods provide post-operative ways to adjust the volume of internal volume occupying device 1750 within a fundoplication to regulate the feelings of satiation within a patient as desired.

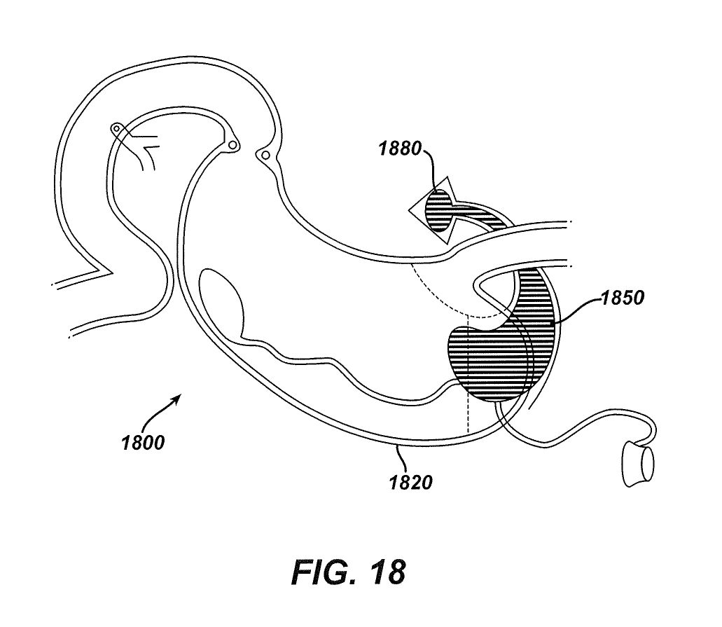

In FIG. 18, a modification of the aforementioned fundoplication procedure incorporating a volume occupying device is shown. In this embodiment, the volume occupying device is a fundal balloon 1850 surgically positioned in a fundus 1820 of a stomach 1800 prior to the fundoplication procedure. However, fundal balloon 1850 includes a retention feature 1880 on its leading end. Retention feature 1880 restricts fundus 1820, making it less likely to be pulled out from the wrapped configuration. The use of a fundal balloon 1850 in this manner offers several benefits and advantages. Fundal balloon 1850 can be used to treat obesity by providing both short-term weight loss and sustained long term excess weight loss and can be adjusted to alter the extent of constriction at the gastro-esophageal junction and thus adjust the amount of weight loss. In other benefits, fundal balloon 1850 can be placed laparoscopically and adjusted via an endoscope in a trans-esophageal procedure. The procedure is also easily reversible by deflation of fundal balloon 1850.

In a second modification of the aforementioned fundoplication procedure, a fundal balloon is incorporated that is controlled by an auto-responsive space occupying device that changes volume as a function of consumption so that the device expands to induce volume reduction and may also apply pressure within the stomach to induce a feeling of satiation when the patient is hungry as previously described. The combination of a fundoplication with auto-responsive space occupying technology produces a device that adjusts the restrictiveness of the fundoplication according to the meal cycle of a patient.

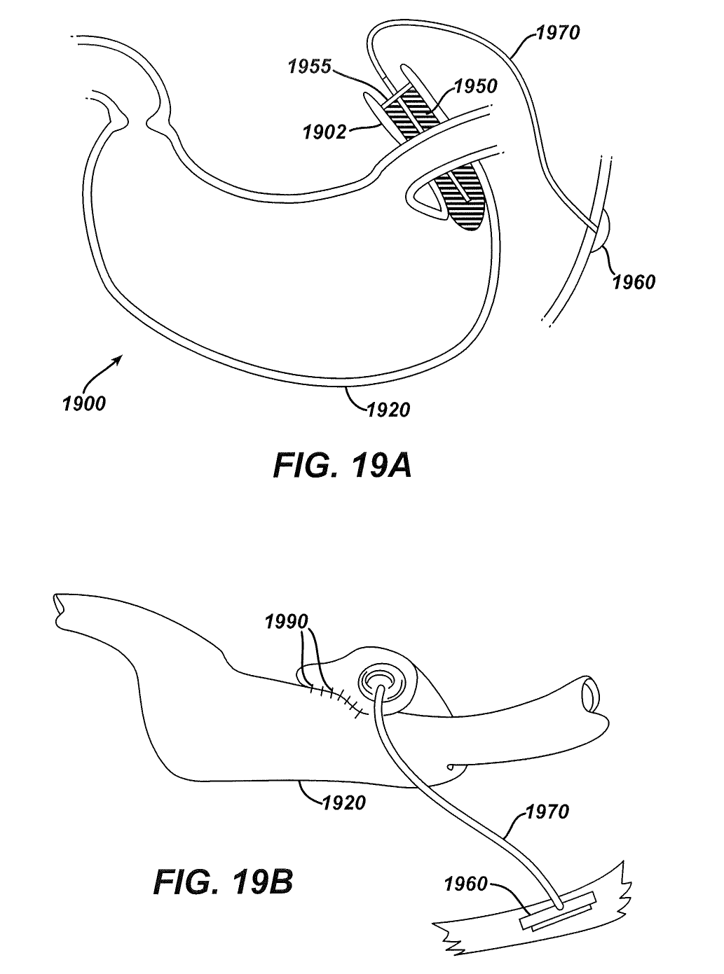

In FIG. 19A, a stomach 1900 is shown having an esophagus 1902. An external volume occupying device 1950 is inserted through esophagus 1902 prior to a fundoplication or partial fundoplication. Volume occupying device 1950 may be an environmental sensitive hydrogel, pH sensitive material, or the like as described above with respect to various auto-responsive embodiments. Volume occupying device 1950 expands or contracts with environmental changes in stomach 1900 corresponding to the eating cycle of the patient. After insertion of volume occupying device 1950, fundus 1920 is then wrapped and placated. Optionally, an intragastric clip 1955 is included with volume occupying device 1950 to aid in securing the position of volume occupying device 1950 in fundus 1920. An external port 1960 may also be used for transcutaneous access to volume occupying device 1950 via a tube or lumen 1970. In FIG. 19B, the completed fundoplication is shown with fundus 1920 placated by sutures 1990 onto anterior side of stomach 1900.

Similarly, external volume occupying device as previous described could be combined with a fundoplication procedure to achieve gastric volume reduction effects. Internal or external ports may be used to regulate the volume of the external devices. The devices may comprise customized geometries to apply pressure selective to different point along the stomach in a fundoplication. As previously described, ports may be incorporated to regulate solution levels (e.g. saline) in the balloon when implanted.

External volume occupying devices used in conjunction with fundoplication procedures offer several benefits to the patient and surgeon. External devices do not require perforation of the stomach lumen and are more easily reversed. Moreover, the devices may be laparoscopically implanted and are post-operatively adjustable based on the needs of the patient.

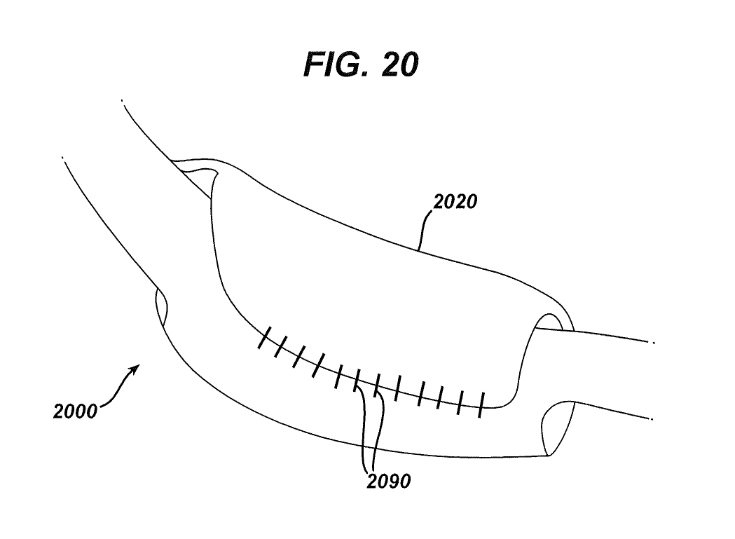

Turning to FIG. 20, another variation on the fundal wrap is presented where a full greater curve wrap extends along the fundoplication to the entire body of the stomach as shown. This may be aided by the placement of a clip (not shown) parallel to the lesser curve of the stomach around which stomach 2000 is wrapped. Generally, the greater curve 2020 of stomach 2000 is first freed before performing this procedure. The procedure can be completed by the closure of the fundoplication with sutures 2090 as shown.

One skilled in the art will appreciate further features and advantages of the invention based on the above-described embodiments. Accordingly, the invention is not to be limited by what has been particularly shown and described. Embodiments provided are presented as exemplary illustrations of the concepts for gastric volume reduction presented herein.

I/We claim:

1. A method for modifying the volume of a stomach, the method comprising:
a. creating a reduced gastric volume passageway through a portion of a stomach through which food can pass; and
b. preventing ghrelin cells within a greater curvature portion of said stomach from contacting said food.

2. The method of claim 1, wherein said reduced gastric volume passageway is formed by passing said greater curvature portion of said stomach through a slot formed in said stomach.

3. The method of claim 1, wherein said reduced gastric volume passageway is formed by passing a portion of a fundus of said stomach through a slot in said stomach.

4. The method of claim 1, wherein said reduced gastric volume passageway is formed by a portion of said stomach wrapped around said passageway.

5. The method of claim 1, wherein said reduced gastric volume passageway functions as a gastric banding mechanism.

6. The method of claim 1, wherein said gastric volume passageway slows the exit of ingested food from said stomach.

7. The method of claim 1, wherein the act of creating a reduced gastric volume passageway comprises:
i. creating more than one slot in said stomach, wherein said more than one slot is oriented horizontally through a fundus portion of said stomach to create multiple fundus portions,
ii. pulling each said fundus portion individually through said more than one slot in said stomach,
iii. wrapping each said fundus portion around said stomach, and
iv. affixing said fundus portions.

8. The method of claim 1, wherein the act of creating a reduced gastric volume passageway comprises:
i. creating a horizontal slot below a portion of said stomach near an entrance to the small intestine, wherein said horizontal slot extends about one half to about two thirds down the length of a fundus of said stomach,
ii. creating a vertical transection from the angle of His along part of a lesser curvature portion of said stomach, sufficient to free up said fundus,
iii. folding said fundus through said horizontal slot and around an antrum of said stomach, and
iv. affixing said fundus.

9. The method of claim 8, further comprising severing the vagus nerve.

10. The method of claim 1, wherein said reduced volume gastric passageway comprises a volume occupying device in a pocket area of a fundus area of said stomach, wherein said fundus area is created by a wrap internal to a wall of said stomach.

11. The method of claim 10, wherein said volume occupying device stretches the wall of said stomach.

12. The method of claim 10, wherein said volume occupying device is selected from at least one of a fundal balloon, an environmental sensitive hydrogel, a pH sensitive material, an auto-adjust band placed between said vertical transection and said angle of His, or a combination thereof.

13. The method of claim 10, wherein said volume occupying device is placed in a location selected from an upper portion of a slot in said stomach and the angle of His, a pocket area of a slot external to said stomach, pressure points within said stomach capable of facilitating feelings of satiation, an outer portion of a wall of said stomach, and combinations thereof.

14. The method of claim 10, wherein said volume occupying device is pH sensitive.

15. The method of claim 1, wherein said method is used to treat or cure a condition selected from at least one of metabolic disorders, obesity, anorexia, depression, insomnia, learning disorders, attention disorders, memory loss, or a combination thereof.

16. A method of regulating the activation of ghrelin hormone within a stomach of a patient, the method comprising modifying ghrelin octanoyl acyl transferase access to ghrelin within the stomach, wherein the act of modifying ghrelin octanoyl acyl transferase access to ghrelin within the stomach comprises creating a fundal wrap; wherein said method results in an effect selected from treatment of weight disorders, promotion of learning and/or memory functions, treatment of stress-induced depression, promotion of healthy sleep, and combinations thereof.

17. The method of claim 16, wherein ghrelin octanoyl acyl transferase interaction with ghrelin is decreased.

18. The method of claim 16, wherein ghrelin octanoyl acyl transferase interaction with ghrelin is increased.

19. The method of claim 16, further comprising the step of implanting a volume occupying device in said stomach.

20. A method of regulating gastric volume, the method comprising the steps of
a. performing a full or partial fundoplication procedure in a patient, wherein the act of performing a full or partial fundoplication procedure comprises attaching one end of a fundus in said patient to an esophagus in said patient; and
b. implanting a volume occupying device external to or within said fundus of said patient concurrent with said fundoplication procedure, such that said volume occupying device is capable of expanding or contracting based on environmental changes in said stomach.
